(19) United States
(12) Patent Application Publication
(10) Pub. No.: US 2004/0068107 A1
(43) Pub. Date: Apr. 8, 2004

(54) Method for the production of pentopyranosly nucleosides

(76) Inventor: Gerhard Kretschmar, Eschborn (DE)

Correspondence Address: CONNOLLY BOVE LODGE & HUTZ, LLP, P O BOX 2207, WILMINGTON, DE, 19899, US

(21) Appl. No.: 10/469,073

(22) PCT Filed: Mar. 7, 2002
(86) PCT No.: PCT/EP02/02536

(30) Foreign Application Priority Data
Mar. 9, 2001 (DE) 10111681.0

(51) Int. Cl. C07H 19/048; C07H 19/00

(52) U.S. Cl. 536/27.2; 536/28.1; 536/55.3

(57) ABSTRACT

The present invention relates to an improved process for the preparation of pentopyranosyl nucleosides, in which a significant improvement and simplification of the process step described in DE-A-19741715 can be achieved. Using the process according to invention, a migration of the 2′-acyl protective group from the 2′-position to the 3′-position of the pentopyranoside is brought about, a catalyst of the formula IVa or IVb being used

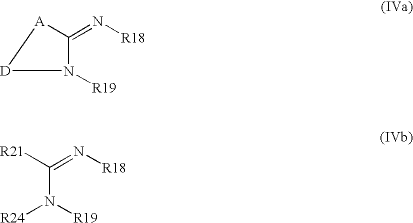

In this formula, A is —CH₂— or —NR²⁰—, R²⁰ is hydrogen, alkyl, cycloalkyl, aryl, aralkyl, each of which is optionally substituted, D is a group of the formula —CₘH₂ₘ—, and

m is an integer from 1 to 6, R¹⁸, R¹⁹ and R²⁴ independently of one another, identically or differently, are hydrogen, alkyl, cycloalkyl, aryl or aralkyl, each of which is optionally substituted, or R¹⁸ and R¹⁹ are together a group —CₒH₂₀—, where o is an integer from 2 to 4, and R²¹ is a group —NR²²R²³, in which R²² and R²³ independently of one another, identically or differently, are hydrogen, alkyl, cycloalkyl, aryl or aralkyl, each of which is optionally substituted.

The present invention relates to an improved process for the preparation of pentopyranosyl nucleosides.

Pentopyranosyl nucleosides and oligomers are a new group of substances which was described for the first time by Eschenmoser et al. (Helv. Chim. Acta 1993, 76, 2161; Helv. Chim. Acta 1995, 78, 1621; Angew. Chem. 1996, 108, 1619-1623). In the course of time, it emerged that the pentopyranosyl nucleosides are a promising aid in the labeling of biomolecules and in the addressing of biomolecules on solid surfaces, such as, for example, in the production of biochips. This development is especially due to the advances in the conjugation of pentopyranosyl nucleosides with biomolecules. In parallel to this, novel processes for the preparation of pentopyranosyl nucleosides and oligomers had to be made available in order to be able to cover the need for these substances.

Such processes are disclosed, in particular, in DE-A-19741715, where pentapyranosyl nucleosides of the formula I or II, their preparation and use are described. Preparation is in this case carried out starting from an unprotected pentopyranoside into which protective groups S_{cl} or S_{c2} are introduced in the 2′-, 3′- or 4′-position, an advantageous synthesis and protective group strategy being followed which makes pentopyranosyl nucleosides accessible in good yields. Moreover, however, the development of alternative processes or the further development and improvement of already existing processes is of great interest in order to increase the availability of the pentopyranosyl nucleosides further.

The object is achieved by the present invention, which is a significant improvement and simplification of the process to give the products of the formulae I and II described in the aforementioned DE-A-19741715. In this process, a migration of the 2′-protective group from the 2′-position to the 3′-position of the pentopyranoside takes place, a catalyst of the formulae IVa and/or IVb being employed.

The present invention thus relates to a process for the synthesis of pentopyranosyl nucleosides, preferably of pentopyranosyl nucleosides of the formula I or II

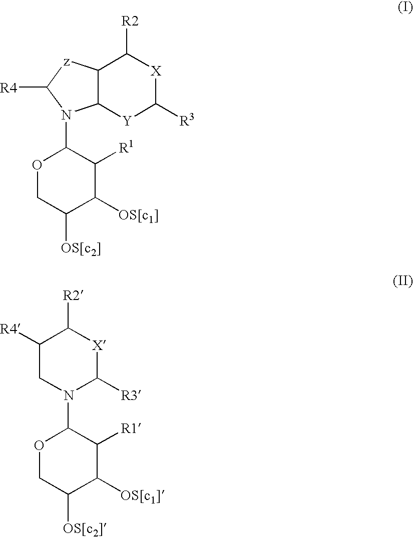

in which

R¹ is hydrogen, —OH, bromine or chlorine,

R², R³ and R⁴ independently of one another, identically or differently, are in each case hydrogen, —NR⁵R⁶, —OR⁷, —SR⁸, ═O, CₙH₂ₙ₊₁ or CₙH₂ₙNR¹⁰R¹¹,

R⁵, R⁶, R⁷ and R⁸ independently of one another, identically or differently, are hydrogen, CₙH₂ₙ₊₁ or CₙH₂ₙ₋₁,

R¹⁰ and R¹¹ independently of one another are hydrogen or CₙH₂ₙ₊₁ or together form a radical of the formula III

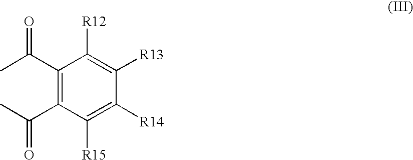

in which R¹², R¹³, R¹⁴ and R¹⁵ independently of one another, identically or differently, are in each case hydrogen, —OR⁷, CₙH₂ₙ₊₁ or CₙH₂ₙ₋₁, —C(O)R⁹, in which R⁷ has the meaning defined above and R⁷ is a linear or branched, optionally substituted alkyl or aryl radical,

X, Y and Z independently of one another, identically or differently, is in each case ═N—, ═C(R¹⁶)— or —N(R¹⁷)— with R¹⁶ and R¹⁷, identically or differently, in each case being hydrogen, CₙH₂ₙ₊₁ or (CₙH₂ₙ)NR¹⁰R¹¹ having the abovementioned meanings, and

S_{c1} is an optionally substituted acyl group, and

S_{c2} is hydrogen or a protective group selected from an optionally substituted acyl, trityl, silyl or allyloxycarbonyl group, and

n in the above formulae is an integer from 1 to 12, preferably 1 to 8 and in particular 1 to 4,

R^{1′} has one of the meanings defined for R¹,

R^{2′}, R^{3′} and R^{4′} independently of one another, identically or differently, are in each case hydrogen, ═O, CₙH₂ₙ₊₁ or —O—CₙH₂ₙ₊₁ or —O—CₙH₂ₙ₋₁ or CₙH₂ₙNR^{10′}R^{11′},

R^{10′} and R^{11′} independently of one another have one of the meanings defined for R¹⁰ and R¹¹,

X′ has one of the meanings defined for X, and

S_{c1′} and S_{c2′} independently of one another have one of the meanings defined for S_{c1} and S_{c2}, comprising the rearrangement of an optionally substituted acyl protective group S_{c1} or S_{c1′} from the 2′-O to the 3′-O atom of the pyranosyl radical in the presence of a catalyst of the formula IVa and/or of the formula IVb

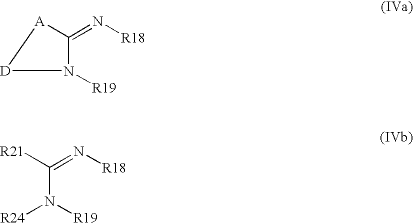

in which A is —CH₂— or —NR²⁰—,

R²⁰ is hydrogen, alkyl, cycloalkyl, aryl, arylalkyl or a polymer radical, in particular a radical of polystyrene, each of which is optionally substituted,

D is a group of the formula —CₘH₂ₘ—, and

m is an integer from 1 to 6,

R¹⁸, R¹⁹ and R²⁴ independently of one another, identically or differently, are hydrogen, alkyl, cycloalkyl, aryl or arylalkyl, each of which is optionally substituted, where R¹⁸ and R¹⁹ can also be linked to one another, by means of which a group —CₒOH₂ₒ— or —CₒH₂ₒ₋₂— where o is an integer from 2 to 4 is preferably obtained and

R²¹ is a group —NR²²R²³, in which R²² and R²³ independently of one another, identically or differently, are hydrogen, alkyl, cycloalkyl, aryl or arylalkyl, each of which is optionally substituted.

Preferred radicals R¹⁸ to R²⁰ and R²² to R²⁴ can contain up to 12, in particular up to 8, carbon atoms. Particularly preferred aryl radicals or cycloalkyl radicals contain 5 to 7 carbon atoms and particularly preferred alkyl radicals contain up to 6 carbon atoms.

An acyl protective group has the general formula —(O)C—R²⁵, in which R²⁵ is an optionally substituted organic radical, such as an alkyl, cycloalkyl, aryl or arylalkyl radical. Aryl radicals can be carbocyclic aromatic or heterocyclic aromatic. Preferably, R²⁵ is alkyl having one to six carbon atoms, phenyl or benzyl.

A trityl protective group has the general formula —C—(R²⁶)₃, in which R²⁶ is an optionally substituted aryl radical, in particular an optionally substituted phenyl radical. Where preferred substituents correspond to those of the radical R²⁵.

A silyl protective group has the general formula —Si(R²³)₃, in which R²⁵ has the abovementioned meaning.

An allyloxycarbonyl protective group has the general formula —(O)C—O—CH₂—CR²⁷═CH₂, in which R²⁷ is an optionally substituted organic radical, such as an alkyl, cycloalkyl, aryl or aralkyl radical, but in particular an alkyl radical having one to six carbon atoms.

The defined protective groups preferably contain up to to 12 carbon atoms, where up to 4 carbon atoms can be replaced by heteroatoms, in particular from the group consisting of N, O, S and P.

In one embodiment, the process according to the invention comprises the steps:

a) introduction of an unprotected pentopyranoside

b) protection of the 2′-position of the pyranosyl radical with an optionally substituted acyl protective group, and, if appropriate, protection of further free positions using protective groups, in particular, if appropriate, protection of the 4′-position of the pyranosyl radical with a protective group S_{c2} or S_{c2}′,

c) removal of a protective group which is optionally located in the 3′-position of the pyranosyl radical and

d) rearrangement of the optionally substituted acyl protective group from the 2′-position to the 3′-position using a catalyst of the formula IVa and/or IVb defined above.

In a particular embodiment, the process according to the invention, in step b), comprises the protection of the 2′- and 4′-positions and optionally the 3′-position simultaneously or in a varied sequence with a protective group, the removal of a protective group which is optionally located in the 3′-position of the pyranosyl radical and then a rearrangement of the protective group S_{c1} or S_{c1}′ from the 2′-position to the unprotected 3′-position using a catalyst of the formula IVa and/or IVb.

Preferably, catalysts of the formula IVa and IVb are employed in which R¹⁸, R¹⁹ and R²⁴ independently of one another, identically or differently, are hydrogen or alkyl having one to six, preferably one to four, carbon atoms or R¹⁸ and R¹⁹ together are a group —(CH₂)ₚ—, where p is an integer from 2 to 4.

Further preferably employed catalysts of the formulae IVa and IVb are compounds in which R²⁰ is hydrogen or alkyl having one to six, preferably one to four, carbon atoms.

Particularly preferred groups A are —CH₂—, —NH—, —N(CH₃)—, —N(C₂H₅)—, —N(C₃H₇)— and —N(C₄H₉)—.

Particularly preferred radicals R²¹ are —N(CH₃)₂, —N(C₂H₅)₂, —N(C₃H₇)₂ or —N(C₄H₉)₂.

Particularly preferred radicals R¹⁸ are hydrogen, CH₃, C₂H₅, C₃H₇ or C₄H₉.

Particularly preferred radicals R¹⁹ are hydrogen, CH₃, C₂H₅, C₃H₇ or C₄H₉.

Particularly preferred radicals R²⁴ are hydrogen, CH₃, C₂H₅, C₃H₇ or C₄H₉.

If R¹⁸ and R¹⁹ in the formulae IVa and IVb make up to a penta-, hexa- or heptacyclic ring, these can be a saturated alkyl or mono- or polyunsaturated alkylene groups.

If, in the above formulae, any radicals are substituted, these are substituents which are inert under the particular reaction conditions.

Preferred examples of these are organic radicals, such as alkyl, cycloalkyl, aryl or aralkyl, preferably having up to 12 carbon atoms, in particular alkyl having one to six carbon atoms, phenyl or benzyl. The radicals can in this case be mono- or polysubstituted, mono- to tetrasubstituted radicals being preferred.

The process according to the invention is explained in greater detail below:

In the key step of the synthesis process to give the products of the formulae I and II described in DE-A-19741715, a migration of the 2′-protective group from the 2′-position to the 3′-position of the pentopyranoside, also described there as a migration reaction, is carried out. In the course of this reaction, the 2′-hydroxyl group is selectively liberated to give R¹═OH [cf. formula I] or to give R^{1′}═OH [cf. formula II], which then, for their part, are available for further reactions. For example, by means of phosphitylation the reactive pentopyranosyl phosphoramidites can thus be obtained, which are needed for the synthesis of the oligomeric pentopyranosyl nucleosides.

The following scheme illustrates this migration reaction or rearrangement as exemplified by aβ-D-ribopyranosyl nucleoside. The migration of an acyl radical (e.g. of a benzoyl group) from the 2′-position to the 3′-position of aβ-D-ribopyranasyl nucleoside (nucleobase B) provided in the 4′-position with the protective group PG (e.g. a trityl group) is shown.

In the process described in DE-A-19741715, a benzoyl group is particularly preferably employed as the migrating acyl group (scheme 1):

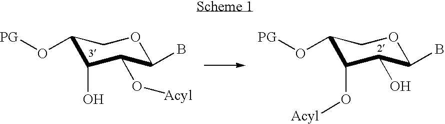

According to DE-A-19741715, this reaction is achieved in a particular embodiment in the presence of a base, in particular in the presence of N-ethyldiisopropylamine and/or triethylamine. At the same time, the reaction can advantageously also be carried out in the same reaction vessel as a one-pot reaction.

Furthermore, it is also advantageous according to DE-A-19741715 if, after the acylation (i.e. after the introduction of the acyl group into the 2′-position) and/or after the migration from the 2′-position to the 3′-position has optionally been carried out, the reaction products are purified by chromatography. Purification after the tritylation is not necessary according to the process in DE-A-19741715, which is particularly advantageous.

The syntheses known from this prior art demand the use of relatively large amounts of chemicals. Furthermore, the reaction times necessary are, as a rule, still very long and the yields of final product are still in need of improvement.

Examples 1 to 4 from DE-A-19741715 can be used as a comparison. In example 1, which describes a synthesis of 1-{3′-O-benzoyl-4′-O-[(4,4′-dimethoxytriphenyl)methyl]-β-D-ribopyranosyl}thymine, the free 4′-position was first tritylated and then benzoylated in the 2′-position. For the migration reaction, relatively large amounts of the following chemicals were then employed:

pyridine (29.7 equivalents), n-propanol (56.0 equivalents), p-nitrophenol (1.58 equivalents), dimethylaminopyridine (0.9 equivalent, DMAP), N-ethyl-diisopropylamine (4.0 equivalents)

The reaction of this mixture was carried out for 48 hours at 61-63° C. and for a further 60 hours at room temperature, i.e. together 4.5 days. Subsequently, the mixture was subjected to aqueous work-up. Then, for prepurification and for subsequent purification, the batch had to be chromatographed on silica gel a further two times. Together, a 48% yield of product were obtained from various fractions.

Analogous procedures and results were described in examples 2 to 4 of DE-A-19741715 (synthesis of N⁴-benzoyl-1-{3′-O-benzoyl-4′-O-[(4,4′-dimethoxytriphenyl)methyl]-β-D-ribopyranosyl}cytosine and of N⁶-benzoyl-9-{3′-O-benzoyl-4′-β-[(4,4′-dimethoxytriphenyl)methyl]-β-D-ribopyranosyl}-adenine and of N⁹-{3′-O-benzoyl-4′-O-[(4,4′-dimethoxytriphenyl)methyl]-β-D-ribopyranosyl}-2-O-allyl-2N-isobutyroylguanine).

The 3-phthalimidylethyl-1-[3′-O-benzoyl4′-O-(4,4′-dimethoxytrityl)-β-D-ribopyranosyl]indole shown below is obtainable in an analogous manner.

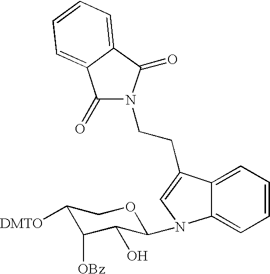

[M=828.92=C₅₁H₄₅N₂O₉]

The process described in DE-A-19741715, and here in particular the process step of the rearrangement of the respective protective group from the 2′-O to the 3′-O group of the pentopyranosyl nucleosides, can be further improved with a view to industrial application.

The desired process improvements are aimed especially at the lowering of the economically and ecologically inexpedient use of large amounts of “ballast chemicals”, their expensive removal by means of column chromatography during product isolation, the improvement of the product yields and the reduction of long reaction times with heating of the reaction mixtures. The latter procedure is especially disadvantageous or possibly even prohibitive in the case of sensitive starting materials or products. These deficiencies are now eliminated in a surprisingly simple manner by the present invention.

It has been found that the excesses of various chemicals, such as pyridine, N-ethyidiisopropylamine or triethylamine, dimethylaminopyridine (DMAP), n-propanol and p-nitrophenol, mentioned according to the teaching of DE-A-19741715, are not needed, since the critical migration reaction of the 2′-O-protected 0-protected compounds, such as the acyl compounds, to give the 3′-O-protected compounds, such as the 3′-O-acyl compounds, can be carried out even without heating under significantly milder conditions, a greatly reduced reaction time, higher yield and frequently without chromatographic purification, by using a catalyst of the formulae IVa and/or IVb defined above.

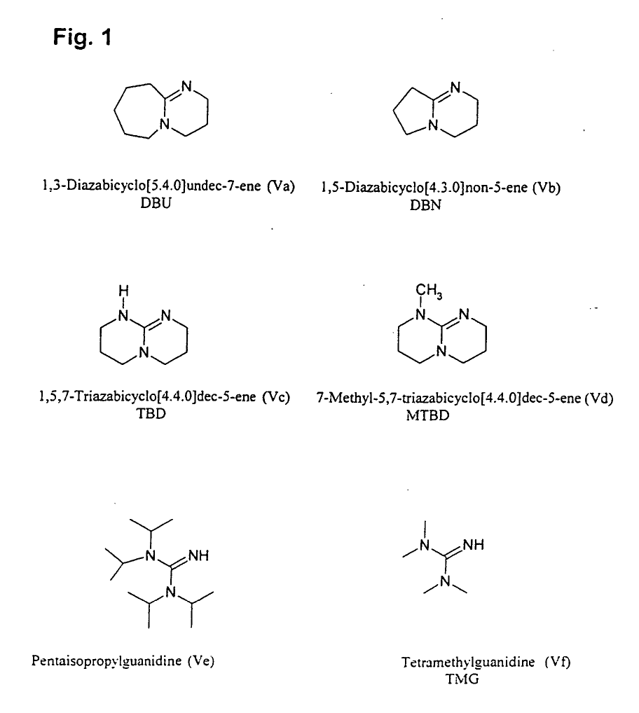

In FIG. 1, some typical examples of catalysts of the formula IVa or IVb are shown (designated there as compounds Va to Vf), which are commercially obtainable and which can be advantageously employed in the process according to the invention. Polymer-bound variants can also be used, such as, for example, polymer-bound TBD (Vc, from Aldrich, number 35,875-4). Polymer-bound catalysts can be removed simply by filtration.

Most other highly active catalysts, such as, for example, Va-d and Vf, can be removed simply by washing the organic reaction solution with water if a water-immiscible solvent has been used or after the water-soluble solvent has been replaced by one of this type.

The catalyst of the formula IVa or IVb can be employed at room temperature in amounts from 0.01 to 20 mole equivalents, preferably in amounts from 0.05 to 10 mole equivalents, particularly preferably in amounts from 0.1 to 0.99 mole equivalent, based on the amount of unprotected starting compound. Equimolar or superstoichiometric amounts, for example 2-10 mole equivalents, can likewise be used, in particular in the case of inexpensive catalysts, if a very rapid reaction is desired.

The reaction is customarily carried out in an inert organic solvent or in suitable solvent mixtures, for example in tetrahydrofuran, methyl or ethyl acetate, N,N-dimethylformamide, dimethyl sulfoxide, diethyl ether, dioxane, acetonitrile, ethylene glycol dimethyl ether, benzene, toluene, dichloromethane or dichloroethane, preferably in an inert, low-boiling solvent in which the reaction components can easily dissolve at room temperature or on gentle warming.

The reaction temperature is customarily 0° C. up to the boiling temperature of the solvent, preferably 150° C. to 40° C., particularly preferably room temperature around 20° C.

The reaction times are in the range from one to a few minutes when using equimolar or superstoichiometric amounts of the catalyst IVa or IVb, up to about 6-12 hours when using 0.01 to 0.99 mole equivalent.

Particularly preferably, the following compounds can be prepared using the process according to the invention:

N⁹-{3′-O-benzoyl-4′-O-[(4,4′-dimethoxytriphenyl)methyl]-β-D-ribopyranosyl}-2-O-allyl-2N-isobutyroylguanine,

N⁶-benzoyl-9-{3′-O-benzoyl-4′-O-[(4,4′-dimethoxytriphenyl)methyl]-β-D-ribopyranosyl}-adenine,

1-{3′-O-benzoyl-4′-O-[(4,4′-dimethoxytriphenyl)methyl]-β-D-ribopyranosyl}thymine,

N⁴-benzoyl-1-{3′-O-benzoyl-4′-O-[(4,4′-dimethoxytriphenyl)methyl]-β-D-ribopyranosyl}cytosine and

3-phthalimidylethyl-1-[3′-O-benzoyl-4′-O-(4,4′-dimethoxytrityl)-β-D-ribopyranosyl]indole.

The following figures and examples are intended to describe the invention in greater detail without restricting it:

### EXAMPLE 1

### Synthesis of 1-{3′-O-benzoyl-4′-O-[(4,4′-dimethoxytriphenyl)methyl]-β-D-ribopyranosyl}thymine (scheme 2)

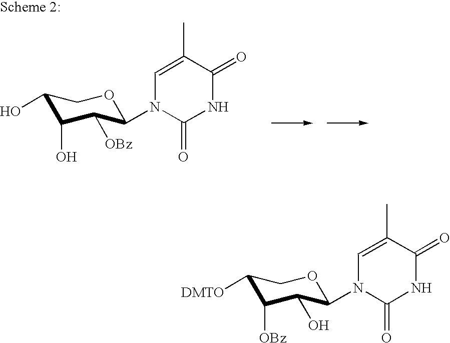

Bz=benzoyl, DMT=dimethoxytrityl

The starting compound 1-{2′-O-benzoyl-4′-O-[(4,4′-dimethoxytriphenyl)-methyl]-βD-ribopyranosyl}thymine was prepared by benzoylation of 1-{4′-O-[(4,4′-dimethoxytriphenyl)methyl]-β-D-ribopyranosyl}thymine with benzoyl chloride in pyridine according to standard processes [¹H NMR (CDCl₃, 400 MHz, ppm): 7.95 (m, 1H, 2″-benzoyl), 6.19 (d, 1H, H-1′), 4.82 (dd, 1 H, H-5a′), 3.37 (m, 1 H, H-5a′)]. 33.0 mg (50 μmol) and 7.0 μl (49.5 μmol) of DBU (Va) in 2 ml of dichloromethane reacted for 12 h at 20° C. DBU was washed out with water, and the org. phase was dried and concentrated.

Yield: 33 mg (100%). According to NMR and HPLC, this product still contained 17.5% of starting material which was still not rearranged. The corrected HPLC yield was accordingly 82.5% [¹H NMR (CDCl₃, 400 MHz, ppm): 8.18 (m, 1H, 2″-benzoyl), 5.92 (m, 1H, H-3′), 5.88 (d, 9.4 Hz, 1H, H-1′), 2.61 (dd,1H, H-5a′)]. The migration product was furthermore compared according to HPLC with an authentic sample according to the process of DE-A-19741715 and found to be identical.

HPLC Conditions:

Merck Lichrosphere 100 column, 5 μm, 4×250 mm; flow rate 1 ml/min; UV detection 210 nm; eluent socratic 41% by volume water, 59% by volume acetonitrile

Retention Times:

Starting material O-2′-benzoate: 13.4 min; product O-3′-benzoate: 12.7 min

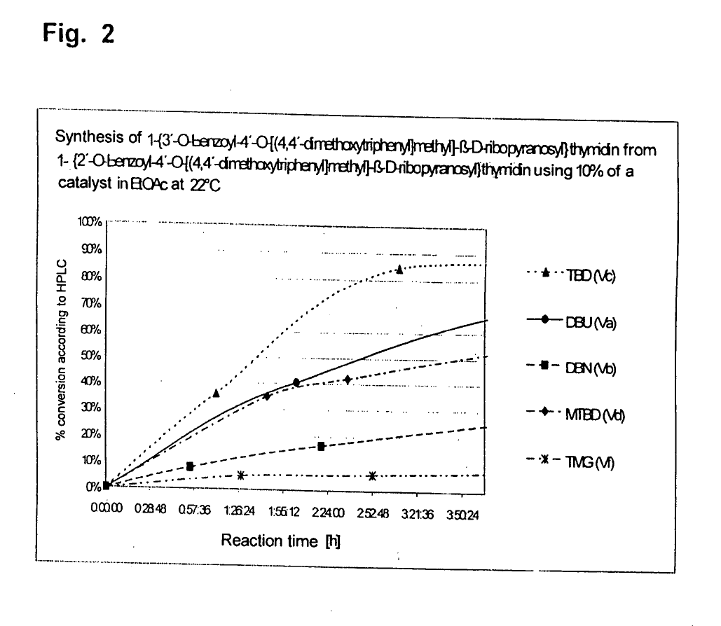

FIG. 2 describes the time conversion of the migration reaction according to HPLC determination. In this process, the conversion of 1-{2′-O-benzoyl4′-O-[(4,4′-dimethoxytriphenyl)methyl]-β-D-ribopyranosyl}thymine to 1-{3′-O-benzoyl-4′-O-[(4,4′-dimethoxytriphenyl)methyl]-β-D-ribopyranosyl}thymine is carried out using 10% of a catalyst of the formula (IV) at 22° C. in ethyl acetate. The activity of various catalysts was investigated, as shown in FIG. 2.

The efficiency of the catalysts investigated is varied. More rapid conversions can of course be achieved using correspondingly larger amounts of (IV). Thus, instead of the relatively expensive TBD (Vc), for example, the relatively inexpensive DBU (Va) or DBN (Vb) can also be employed successfully for preparative purposes in somewhat larger amounts.

### EXAMPLE 2

### Synthesis of N⁴-benzoyl-1-{3′-O-benzoyl-4′-O-[(4,4′-dimethoxytriphenyl)-methyl]-β-D-ribopyranosyl}cytosine (scheme 3)

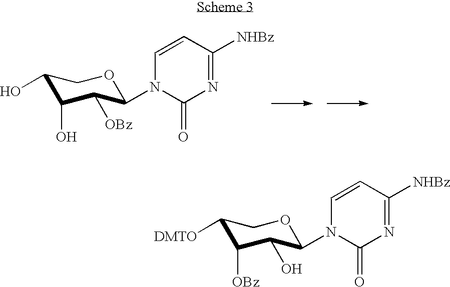

Bz=benzoyl, DMT=dimethoxytrityl

The starting compound for the migration reaction, N⁴-benzoyl-1-{2′-O-benzoyl-4′-O-[(4,4′-dimethoxytriphenyl)methyl]-β-D-ribopyranosyl}cytosine can be obtained in 51% yield either according to DE-A-19741715 (example 2 therein), or better, with complete avoidance of the expensive column chromatography, as a crystalline pure product in 46% total yield as follows:

199 g (0.57 mol) of N⁴-benzoyl-1-β-D-ribopyranosylcytosine, 230 ml (132.4 mmol; 2.3 eq.) of anisaldehyde dimethyl acetal and 2.5 g (13.1 mmol) of p-toluenisulfonic acid were suspended in 2000 ml of N,N-dimethylformamide. 200 ml of solvent were distilled off in vacuo. After stirring at a bath temperature of 70° C. (70 mbar) for 1 hour, a further 2.5 g (13.1 mmol) of p-toluenesulfonic acid were added and stirring was continued for a further 1 hour at a bath temperature of 70° C. and 70 mbar. The solvent was distilled off in vacuo, and the residue was taken up in 650 ml of pyridine and treated with 100 ml (0.82 mol; 1.4 eq.) of benzoyl chloride. After stirring for 12 h at room temperature, the solvent was distilled off in vacuo and the residue was filtered through silica gel using dichloromethane. After concentration, 428 g of a crude product were obtained, which was further processed as follows: 190.0 g thereof (theoretically at most 253 mmol) were dissolved in 2396 ml of methanol and 218.4 ml (2835 mmol, 8.50 eq.) of trifluoroacetic acid were rapidly added dropwise in an ice bath. The ice bath was removed after 10 min. After a further 80 min, the mixture was treated cautiously with 238.2 g (2835 mmol, 8.5 eq.) of solid sodium hydrogencarbonate. After stirring for 5 min, the neutralized solution was treated with 2000 ml of water and extracted with 2000 ml of dichloromethane. The water phase was extracted a further 2 times with 1300 ml of dichloromethane each time, and the collected organic phases were rapidly dried using sodium sulfate and concentrated down to about 1 liter. In a refrigerator first 27.8 g and, from the mother liquor (after washing with water and drying), a further 24.7 g of product crystallized. Total yield of N⁴-benzoyl-1-[(2′-O-benzoyl)-β-D-ribopyranosyl]cytosine: 52.5 g (116.3 mmol; 46% based on N⁴-benzoyl-1-β-D-ribopyranoslylcytosine employed).

The tritylation and migration reaction according to the present process according to the invention were not carried out as a one-pot reaction as recommended in DE-A-19741715 (example 2 therein), since the desired final product can be isolated more expediently in pure form without column chromatography. This especially applies for the reason that after the tritylation, which occurs virtually quantitatively, all excess reagents could already be separated off by simple precipitation of the intermediate and thus chromatography was also no longer necessary after the migration reaction:

39.44 ml (230.3 mmol, 2.0 eq.) of N-ethyldiisopropylamine were added dropwise under argon and with stirring to 52.00 g (115.1 mmol) of N⁴-benzoyl-1-[(2′-O-benzoyl)βp-D-ribopyranosyl]cytosine, 572 ml of dry dichloromethane, 270 ml of dry pyridine, 78.06 g (230.3 mmol, 2.0 eq.) of dimethoxytrityl chloride and 1.56 g (11.5 mmol, 0.1 eq.) of DMAP. After 4 h, the mixture was treated with saturated NaHCO₃ solution, stirred for 5 min, the organic phase was separated off, the water phase was extracted by shaking with dichloromethane a further 2 times and the combined organic phases were dried over magnesium sulfate. After concentration with toluene (removal of pyridine), 140 g of yellow oil were obtained, which was dissolved in a little dichloromethane and added dropwise with stirring to 5 l of iso-hexane. The yellow product was filtered off with suction, washed with iso-hexane and dried at 45° C. in vacuo.

Yield: 90 g (100%) of N⁴-benzoyl-1-{2′-O-benzoyl4′-O-[(4,4′-dimethoxytriphenyl)methyl]-β-D-ribopyranosyl}cytosine, which was dissolved in 900 ml of ethyl acetate and treated with 224 ml (0.10 mol, 0.87 eq. of DBU (Va). In the course of this, the solution immediately turned violet. After stirring at room temperature for 3 h, the reaction was complete according to HPLC analysis. As shown in example 2, it is not necessary to use the excess amount of (Va), the reaction can just as well be allowed to stand with 10 mol % of reagent overnight. Using the excess employed here and because of the simple removal of (Va), the reaction time, however, could be greatly reduced. The reaction batch was worked up by washing the solution with 5 times 200 ml of water each time until neutral, then drying with sodium sulfate and concentrating. The residue was dissolved in 180 ml of dichloromethane at 80° C. and slowly treated with 900 ml of tert-butyl methyl ether until the clear solution slowly became cloudy. After cooling, the crystalline product was filtered off with suction and dissolved again in 270 ml of dichloromethane. After addition of 270 ml of tert-butyl methyl ether, crystals were rapidly obtained in the cold, which were filtered off with suction and dried at 40° C. in vacuo.

Yield of pure N⁴-benzoyl-1-{3′-O-benzoyl-4′-O-[(4,4′-dimethoxytriphenyl)-methyl]-β-D-ribopyranosyl}cytosine (HPLC purity <99%): 55.3 g (73.4 mmol) of colorless crystals. The mother liquor was extracted by stirring with diethyl ether and the insoluble portion was reacted and worked up again in a manner analogous to that described above. 5.5 g of a further product fractions could be obtained by this means. This corresponds to a total yield of 60.8 g (81.4 mmol, 70.5% over 2 stages).

For the comparison with the prior art: Corresponding to example 2 of DE-A-19741715, on a comparable scale it was possible to obtain only a 51% product yield after boiling for a number of days and chromatography on a large column (50×10 cm). ¹H NMR (CDCl₃, 400 MHz, ppm): 8.85 (brm, 1H, NH), 6.03 (d, 9.8 Hz, 1H, H-1′), 5.88 (m, 1H, H-3′), 4.14 (br, 1h, OH), 3.92 (ddd, 1H, H-4′), 3.76, 3.77 (2s, 6H, OMe), 3.66-3.76 (m, 2H, H-2′, H-5a′), 2.72 (dd, 11 Hz, 5 Hz, 1H, H-5b′). ¹³C NMR (DEPT, CDCl₃, ppm): 55.26 (OMe), 66.00 (C -5′), 67.90 C-4′), 71.18 (C-3′), 73.96 (C-2′), 82.26 (C-1′), 87.30 (trityl-C). The structure of the migration product was also identical according to HPLC with an authentic sample according to example 2 of DE-A-19741715.

### EXAMPLE 3

Reaction course in the synthesis of N⁴-benzoyl-1-{3′-O-benzoyl-4′-O-[(4,4′-dimethyoxytriphenyl)methyl]-β-D-ribopyranosyl}cytosine using catalysts of the formula (IV).

In a manner analogous to that described in example 1, different conversion/time courses were determined. It was possible to show here, for example, that using 10% DBU (Va), TBD (Vc) or DBN (Vb), the reaction is almost finished after less than eight hours at room temperature

HPLC Conditions:

Merck Lichrosphere 100 column, 5 μm, 4×250 mm; flow rate 1 ml/min; UV detection 210 nm; gradient in 13 min from 70% B to 82% B, where eluent A: 95% by volume water, 5% by volume acetonitrile and eluent B: 95% by volume acetonitrile, 5% by volume water.

Retention times: starting material O-2′-benzoate: 8.9 min; product O-3′-benzoate: 8.3 min.

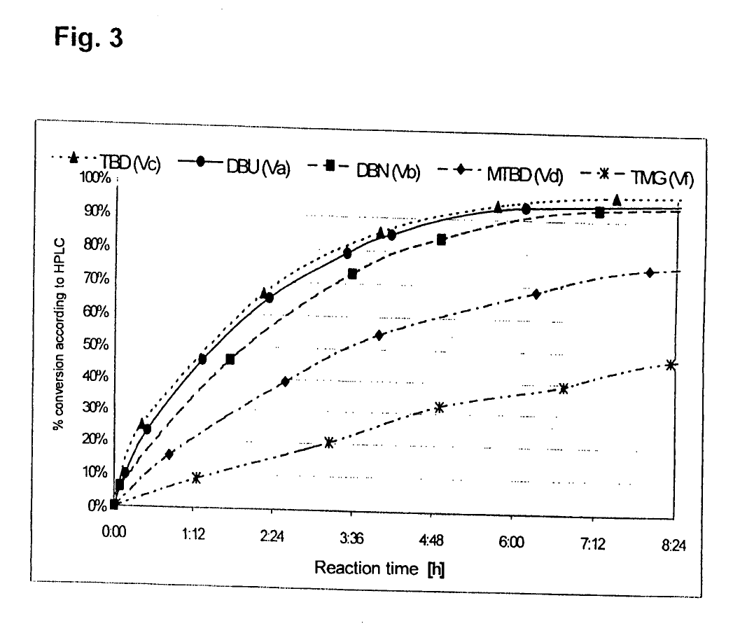

FIG. 3 shows the results of the conversion of 1-{2′-O-benzoyl-4′-O-[(4,4′-dimethoxytriphenyl)methyl]-β-D-ribopyranosyl)cytosine to 1-{3′-O-benzoyl-4′-O-[(4,4′-dimethoxytriphenyl)methyl)-β-D-ribopyranosyl}cytosine using 10% of a catalyst of the formula (IV) at 22° C. in ethyl acetate.

### EXAMPLE 4

### Conversion of N⁴-dibenzoyl-9-{2′-O-benzoyl-4′-O-[(4,4′-dimethoxytriphenyl)methyl]-β-D-ribopyranosyl}adenine using DBU (IIIa) to N⁶-dibenzoyl-9-{3′-O-benzoyl-4′-O-[(4,4′-dimethoxytriphenyl)methyl]-β-D-ribopyranosyl}adenine

(Scheme 4)

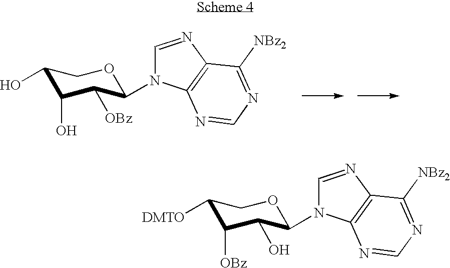

Bz=benzoyl, DMT=dimethoxytrityl

The reaction is carried out analogously to example 1.

HPLC Conditions:

Merck Lichrosphere 100 column, 5 μm, 4×250 mm; flow rate 1 ml/min; UV detection 210 nm; gradient in 13 min from 70% B to 82% B, where eluent A: 95% by volume water, 5% by volume acetonitrile and eluent B: 95% by volume acetonitrile, 5% by volume water.

Retention times: starting material O-2′-benzoate: 13.0 min; product O-3′-benzoate: 13.6 min.

N⁶-Dibenzoyl-9-{2′-O-benzoyl-4′-O-[(4,4′-dimethoxy-triphenyl)methyl]-β-D-ribopyranosyl}adenine (authentic sample according to DE-A-19741715) and 1 mole equivalent of DBU (Va) were reacted at 22° C. in ethyl acetate. HPLC analysis after 1 h showed conversion to 89% 3′-O-benzoate and 11 % residual 2′-O-benzoate.

### EXAMPLE 5

### Synthesis of 9-{3′-O-benzoyl4′-O-[(4,4′-dimethoxytriphenyl)methyl]-β-D-ribopyranosyl}-2-O-allyl-2-N-isobutyroylguanine (scheme 5)

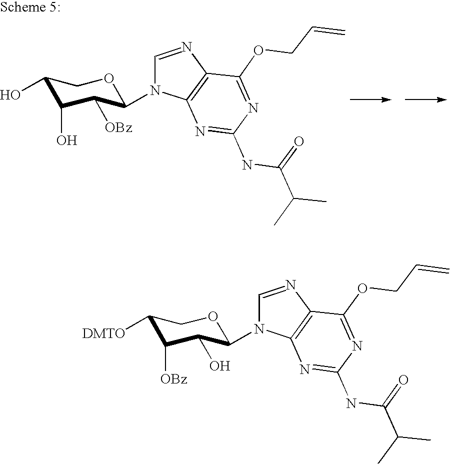

Bz=benzoyl, DMT=dimethoxytrityl

The synthesis of the precursor 9-{2′-O-benzoyl4′-O-[(4,4′-dimethoxytriphenyl)methyl]-β-D-ribopyranosyl}-2-O-allyl-2-N-isobutyroyl-guanine was carried out according to an improved method analogously to the process described in example 2 for the cytosine structural unit.

Thus, 0.200 g (0.246 mmol) of crude 9-{2′-O-benzoyl-4′-O-[(4,4′-dimethoxytriphenyl)methyl]-β-D-ribopyranosyl}-2-O-allyl-2-N-isobutyroylguanine [C₄₆H₄₇N₅O₉; 813.9], which was still impure according to TLC and HPLC, and 0.64 ml (3.09 mmol) of DBU (Va) were dissolved in ethyl acetate (2 ml) at 22° C. According to HPLC and TLC (dichloromethane/ethyl acetate 5:1), the product and starting material were present in the ratio about 90:10 virtually immediately after dissolution of the components. The solution was washed 5 times with water until the pH was neutral, dried and concentrated, and the product was purified on silica gel (dichloromethane/ethyl acetate 10:1 to 6:1 with 1% triethylamine). 0.110 mg (55%, based on the impure starting material) of 9-{3′-O-benzoyl-4′-O-[(4,4′-dimethoxytriphenyl)methyl]-β-D-ribopyranosyl}-2-O-allyl-2-N-isobutyroylguanine and about 90 mg of the impurities already present in the starting material, which eluted at relatively high RF, were thus obtained.

The starting material was barely still detectable after chromatography, such that the yield was between 55% and 100% based on the starting material effectively employed.

The reaction could also be carried out using catalytic amounts of DBU (Va). Thus, the corresponding batch using 14.68 μl (0.098 mmol), i.e. 0.4 mole equivalents of DBU (Va), produced the same result after a reaction time of 75 min. In a further experiment using only 0.02 mole equivalent of DBU (Va), a conversion of 50% after 2 hours was determined by means of HPLC.

¹H NMR (CDCl₃, 400 MHz, ppm): 2.62, 2.64 (2d, 6H, 2 Me), 3.77, 3.78 (2s, 6H, 2 OMe), 5.66 (d, 9.5 Hz, 1 H, H-1′).

### EXAMPLE 6

### Comparison Experiments

Conversion of 1-{2′-O-benzoyl-4′-O-[(4,4′-dimethoxytriphenyl)methyl)-β-D-ribopyranosyl}cytosine to 1-{3′-O-benzoyl-4′-O-[(4,4′-dimethoxytriphenyl)-methyl)-β-D-ribopyranosyl}cytosine with the chemicals used in DE-A-19741715 in the preferred embodiment under the reaction conditions of the present invention, i.e. in particular at room temperature and relatively short reaction times.

Conversion of 1-{2′-O-benzoyl-4′-O-[(4,4′-dimethoxytriphenyl)methyl)-β-D-ribopyranosyl}cytosine, 0.25 g (0.332 mmol) using

a)

N-ethyldiisopropylamine 5.68 μl (0.033 mmol, 0.1 equivalent) in 2.5 ml of ethyl acetate.

b)

triethylamine 4.62 μl (0.033 mmol, 0.1 equivalent) in 2.5 ml of ethyl acetate

c)

N-ethyldiisopropylamine 5.68 μl (0.033 mmol, 0.1 equivalent) in 2.5 ml of ethyl acetate

triethylamine 4.62 μl (0.033 mmol, 0.1 equivalent)

d)

using all of the reaction components mentioned in DE-A-19741715, example 2, in corresponding quantitative ratios

pyridine (2.7 ml)

dimethylaminopyridine 40.52 mg (DMAP, 0.332 mmol, 1 equivalent)

triethylamine 138.5 μl (0.995 mmol, 3 equivalents)

p-nitrophenol 92.27 mg (0.663 mmol, 2 equivalents)

n-propanol 498.3 μl (6.633 mmol, 20 equivalents)

Result: In all experiments a-c, no conversion to the desired 3′-O-benzoate was found by means of HPLC after stirring for 24 h at room temperature. The conversion was <3% in the experiment. It follows from this that the desired migration reaction of the 2′-O-benzoate to the 3′-O-benzoate can be achieved neither by the individual components nor by the entire mixture of the embodiment particularly preferred in DE-A-19741715 under the comparatively mild reaction conditions and high conversions as in the present invention.

1. A process for the synthesis of pentopyranosyl nucleosides of the formula I or II 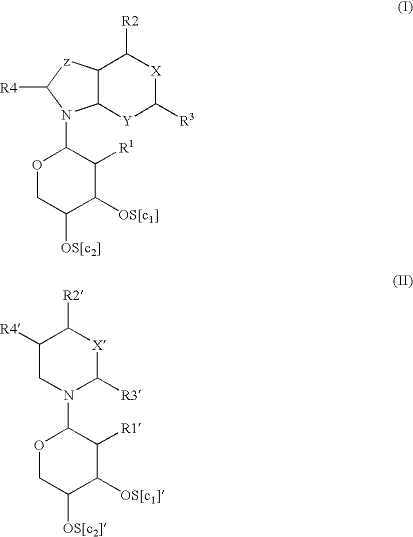
in which
R¹ is hydrogen, —OH, bromine or chlorine,
R², R³ and R⁴ independently of one another, identically or differently, are in each case hydrogen, —NR⁵R⁶, —OR⁷, —SR⁸, ═O, CₙH₂ₙ₊₁ or CₙH₂ₙNR¹⁰R¹¹,
R⁵, R⁶, R⁷ and R⁸′ independently of one another, identically or differently, are hydrogen, CₙH₂ₙ₊₁ or CₙH₂ₙ₋₁,
R¹⁰ and R¹¹ independently of one another are hydrogen or CₙH₂ₙ₊₁ or together form a radical of the formula III 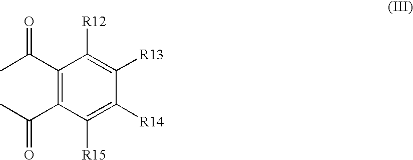
in which R¹², R¹³ , R¹⁴ and R¹⁵ independently of one another, identically or differently, are in each case hydrogen, —OR⁷, CₙH₂ₙ₊₁ or CₙH₂ₙ₋₁, —C(O)R⁹, in which R⁷ has the meaning defined above and
R⁹ is a linear or branched alkyl or aryl radical, which can be present in mono - or polysubstituted form,
X, Y and Z independently of one another, identically or differently, is in each case ═N—, ═C(R¹⁶)— or —N(R¹⁷)— with R¹⁶ and R¹⁷, identically or differently, in each case being hydrogen, CₙH₂₊₁ or (CₙH₂ₙ)NR¹⁰R¹¹ having the abovementioned meanings, and
S_{c1} is an acyl group, which can be mono- or polysubstituted, and
S_{c2} is hydrogen or a protective group selected from an acyl, trityl, silyl or allyloxycarbonyl group, where the protective group can be mono- or polysubstituted, and
n in the above formulae is an integer from 1 to 12,
R^{1′} has one of the meanings defined for R¹,
R^{2′}, R^{3′} and R^{4′} independently of one another, identically or differently, are in each case hydrogen, ═O, CₙH₂₊₁ or —O—CₙH₂ₙ₊₁ or —O—CₙH₂ₙ₋₁ or CₙH₂ₙNR¹⁰′R^{11′},
R^{10′} and R^{111′} independently of one another have one of the meanings defined for R¹⁰ and R¹¹,
X′ has one of the meanings defined for X, and
S_{c1′} and S_{c2′} independently of one another have one of the meanings defined for S_{c1′} and S_{c2′},
comprising the rearrangement of an acyl protective group S_{c1} or S_{c2}, from the 2′-O to the 3′-O atom of the pyranosyl radical in the presence of a catalyst of the formula IVa and/or of the formula IVb 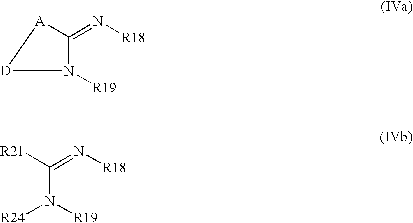
in which A is —CH₂— or —NR²⁰—,
R²⁰ is hydrogen, alkyl, cycloalkyl, aryl, arylalkyl or a polymer radical, which can be mono- or polysubstituted,
D is a group of the formula —CₘH₂ₘ—, and
m is an integer from 1 to 6,
R⁸, R¹⁹ and R²⁴ independently of one another, identically or differently, are hydrogen, alkyl, cycloalkyl, aryl or aralkyl, which can be mono- or polysubstituted, where R¹⁸ and R¹⁹ can also be linked to one another, and
R²¹ is a group —NR²²R²³, in which R²² and R²³ independently of one another, identically or differently, are hydrogen, alkyl, cycloalkyl, aryl or aralkyl, which can be mono- or polysubstituted.

2. The process as claimed in claim 1, comprising the steps:
a) introduction of an unprotected pentopyranoside
b) protection of the 2′-position of the pyranosyl radical with an optionally substituted acyl protective group S_{c1} or S_{c1′}, and, if appropriate, protection of further free positions using protective groups, in particular, if appropriate, protection of the 4′-position of the pyranosyl radical with a protective group S_{c2} or S_{c2′},
c) removal of a protective group which is optionally located in the 3′-position of the pyranosyl radical and
d) rearrangement of the optionally substituted acyl protective group from the 2′-position to the 3′-position using a catalyst of the formula IVa and/or IVb.

3. The process as claimed in claim 2, in which in step b), the 2′- and 4′-positions and optionally the 3′-position are protected simultaneously or in a varied sequence with a protective group, the protective group which is optionally located in the 3′-position of the pyranosyl radical is removed and then a rearrangement of the protective group S_{c1} or S_{c1′} from the 2′-position to the unprotected 3′-position using a catalyst of the formula IVa and/or IVb is carried out.

4. The process as claimed in claim 1, in which R¹⁸, R¹⁹ and R²⁴ independently of one another, identically or differently, is hydrogen or alkyl having one to four carbon atoms or R¹⁸ and R¹⁹ together are a group —(CH₂)ₚ—, where p is an integer from 2 to 4.

5. The process as claimed in claim 1, in which R²⁰ is hydrogen or alkyl having one to four carbon atoms.

6. The process as claimed in claim 1, in which A is —CH₂—, —NH—, —N(CH₃)—, —N(C₂H₅)—, —N(C₃H₇)— and —N(C₄H₉)—.

7. The process as claimed in claim 1, in which R²¹ is —N(CH₃)₂, —N(C₂H₅)₂, —N(C₃H₇)₂ or —N(C₄H₉)₂.

8. The process as claimed in claim 1, in which R¹⁸ is hydrogen, —CH₃, —C₂H₅, —C₃H₇ or C₄H₉.

9. The process as claimed in claim 1, in which R¹⁹ is hydrogen, —CH₃, —C₂H₅, —C₃H₇ or C₄H₉.

10. The process as claimed in claim 1, in which R²⁴ is hydrogen, —CH₃, —C₂H₅, —C₃H₇ or C₄H₉.

11. The process as claimed in claim 1, in which the catalyst of the formula (IVa) or (IVb) is employed in amounts from 0.01 to 20 mole equivalents, based on the unprotected pentopyranoside.

12. The process as claimed in claim 1, in which the reaction is carried out in an inert organic solvent selected from the group consisting of tetrahydrofuran, methyl acetate, ethyl acetate, N,N-dimethylformamide, dimethyl sulfoxide, diethyl ether, dioxane, acetonitrile, ethylene glycol dialkyl ether, benzene, toluene, dichloromethane, methyl isobutyl ketone, chloroform or dichloroethane or in their mixtures.

13. The process as claimed in claim 1, in which the reaction temperature is between 15° C. and 40° C.

14. The process as claimed in claim 1, in which the reaction time is between one minute and 12 hours.

15. The process as claimed in claim 1, where the catalysts are selected from the group consisting of
1,3-diazabicyclo[5.4.0]undec-7-ene in (DBU),
1,5-diazabicyclo[4.3.0.]non-5-ene (DBN),
1,5,7-triazabicyclo[4.4.0]dec-5-ene (TBD),
7-methyl-,5,7-triazabicyclo[4.4.0]dec-5-ene (MTBD),
pentaisopropylguanidine (PIG) or
tetramethylguanidine (TMG).

16. The process as claimed in claim 1 for the synthesis of N⁹-{3′-O-benzoyl-4′-O-[(4,4′-dimethoxytriphenyl)methyl]-β-D-ribo-pyranosyl}-2-O-allyl-2N-isobutyroylguanine,
N⁶-benzoyl-9-{3′-O-benzoyl-4′-O-[(4,4′-dimethyoxyrriphenyl)methyl]-β-D-ribopyranosyl}adenine,
1-{3′-O-benzoyl-4′-O-[(4,4′-dimethoxytriphenyl)methyl]-β-D-ribo-pyranosyl}thymine,
N⁴-benzoyl-1-{3′-O-benzoyl-4′-O-[(4,4′-dimethyoxytriphenyl)methyl]-β-D-ribopyranosyl}cytosine and
3-phthalimidylethyl-1-[3′-O-benzoyl-4′-O-(4,4′-dimethoxytrityl)-β-D-ribopyranosyl]indole.
